(19) United States
(12) Patent Application Publication
(10) Pub. No.: US 2014/0069202 A1
(43) Pub. Date: Mar. 13, 2014

(54) DETECTION OF MECHANICAL STRESS ON COATED ARTICLES

(71) Applicant: Thomas E. Fisk, Green Valley, AZ (US)

(72) Inventor: Thomas E. Fisk, Green Valley, AZ (US)

(21) Appl. No.: 14/003,095

(22) PCT Filed: Mar. 14, 2012
(86) PCT No.: PCT/US12/29089

§ 371 Date: Nov. 20, 2013

Related U.S. Application Data
(60) Provisional application No. 61/452,518, filed on Mar. 14, 2011.

(51) Int. Cl. A61M 5/50; G01L 1/24

(52) U.S. Cl. CPC A61M 5/5086; G01L 1/24
USPC 73/762; 604/111

(57) ABSTRACT

A medical device comprising a wall, a coating of SiOₓ, and a piezochromic material is disclosed. The piezochromic material is associated with the wall, and changes its appearance when the wall is exposed to mechanical stress exceeding a threshold intensity. Also disclosed is a method of interrogating a closed medical device for processing damage, comprising at least the acts of providing a closed medical device and inspecting the medical device. The medical device is inspected from the exterior for a change in the appearance of at least some of its piezochromic material that is characteristic of exposure of the wall to mechanical stress exceeding a threshold intensity greater than zero. Optionally in any embodiment inspecting is carried out using a spectrophotometer to determine the change in the color of at least some of its piezochromic material.

### CROSS REFERENCE TO RELATED APPLICATIONS

This application claims the priority of U.S. Ser. No. 61/452,518, filed Mar. 14, 2011, which is incorporated here by reference.

### STATEMENT REGARDING FEDERALLY SPONSORED RESEARCH OR DEVELOPMENT

[Not Applicable]

### BACKGROUND OF THE INVENTION

The invention concerns the use of mechanical stress detection using piezochromic materials to determine if during the commercial processing of articles, for example medical devices, for example vessels or catheters, for example plastic medical vials, sample vessels, syringe barrels, or micro-titer plates, a strain has been imparted on the article. The ability to detect whether a strain has been imparted on the plastic article is particularly desirable if a thin, high modulus coating or layer is present on the plastic.

Glass is the predominate material utilized in parenteral vials and syringe barrels. During processing operations including washing, filling, sterilization, and packaging, individual glass articles can become misaligned with automated handling machines operating at high speeds, resulting in high impact shearing and compression forces on the article. Due to the brittle nature of glass, these forces frequently result in catastrophic failure of the part, e.g. complete breakage of the article.

Glass-like, nano-thin plasma barrier coated plastic substrates are under consideration for use in medical devices including parenteral vials and syringe barrels. The strain-to-break for these barrier coating or layers is typically three to five percent, whereas the strain-to-break for plastic articles is much higher than glass. Thus, if a coated plastic article is strained under similar process conditions as utilized with glass articles, it is possible the plastic article might deform and reform its shape, but not catastrophically fail (break); this same stress might very well cause failure (cracking) of vessel wall or the glass-like coating or layer. While assessment of the integrity of the glass-like coating or layer on the plastic article is readily accomplished after coating in an empty state, current methods do not readily permit assessment of the integrity of the glass-like coating or layer once filled with payload contents and ready for distribution.

U.S. Pat. Nos. 5,501,945; 6,108,475; and 7,682,696; Kunzelman, Jill Nicole: Polymers With Integrated Sensing Capabilities (Doctoral Thesis, Case Western Reserve University, 2009); and Characterization of the Piezochromic Behavior of Some Members of the CuMo₁₋ₓWₓO₄ Series, Inorg. Chem., 2008, 47 (7), pp 2404-2410, might be pertinent.

### SUMMARY OF THE INVENTION

The present inventor has found that the strain imparted on an article, for example but not limited to a medical device or vessel, can be assessed by associating the vessel with a piezochromic indicator. An aspect of the invention concerns an article comprising a wall, a coating or layer of SiOₓ, and a piezochromic material. The wall optionally has an interior surface defining a lumen and an exterior surface. The coating or layer is optionally located on the interior surface, and optionally visible by inspection of or through the exterior surface. The piezochromic material is associated with the wall. The piezochromic material has the property of changing its appearance when the wall is exposed to mechanical stress exceeding a threshold intensity.

Another aspect of the invention concerns a method of interrogating a closed vessel for processing damage, comprising at least the acts of providing a vessel and inspecting the vessel. The vessel is inspected from the exterior for a change in the appearance of at least some of its piezochromic material that is characteristic of exposure of the wall to mechanical stress exceeding a threshold intensity.

Optionally in any embodiment at least a portion of the coating or layer is a barrier coating or layer.

Optionally in any embodiment at least a portion of the coating or layer has the ratio of elements SiOₓ, in which x in this formula is from about 1.5 to about 2.9.

Optionally in any embodiment at least a portion of the coating or layer is applied using chemical vapor deposition.

Optionally in any embodiment at least a portion of the coating or layer is applied using plasma enhanced chemical vapor deposition

Optionally in any embodiment at least a portion of the coating or layer has a thickness of less than 200 nm.

Optionally in any embodiment at least a portion of the wall is comprised of thermoplastic material.

Optionally in any embodiment at least a portion of the piezochromic material is coated on at least a portion of the exterior surface.

Optionally in any embodiment at least a portion of the piezochromic material is a layer between the interior surface of the wall and at least a portion of the coating or layer.

Optionally in any embodiment at least a portion of the piezochromic material is incorporated in the wall.

Optionally in any embodiment at least a portion of the piezochromic material is homogeneously incorporated in the wall.

Optionally in any embodiment the piezochromic material comprises:
a triaryl imidazole dimer of bis-2,4,5-triaryl imidazole;
bis-tetraaryl pyrrole;
a bianthrone;
xanthylidene anthrone;
dixanthylene;
helianthrone;
a piezochromic compound having the formula: CuMo₁₋ₓWₓO₄; or
a combination of two or more of these.

Optionally in any embodiment the piezochromic material comprises a triaryl imidazole dimer of bis-2,4,5-triaryl imidazole.

Optionally in any embodiment each aryl moiety is independently selected from phenyl, p-tolyl, p-chlorophenyl, and p-anisyl.

Optionally in any embodiment the piezochromic material comprises:
2,2′,4,4′5,5′-hexaphenyl bisimidazole;
2,2′,4,4′5,5′-hexa-p-tolyl bisimidazole;
2,2′,4,4′5,5′-hexa-p-chlorophenyl bisimidazole;
2,2′-di-p-chlorophenyl-4,4′,5,5′ tetraphenyl bisimidazole;
2,2′-di-p-anisyl-4,4′,5,5′-tetraphenyl bisimidazole;
2,2′-di-p-tolyl-4,4′,5,5′-tetraphenyl bisimidazole or
a combination of two or more of these.

Optionally in any embodiment the piezochromic material comprises a bis-tetraaryl pyrrole.

Optionally in any embodiment the piezochromic material comprises bis-tetraphenylpyrrole.

Optionally in any embodiment the piezochromic material comprises a bianthrone.

Optionally in any embodiment the piezochromic material comprises Δ10,10′-bianthrone.

Optionally in any embodiment the piezochromic material comprises 2,4,2′,4′-tetramethylbianthrone.

Optionally in any embodiment the piezochromic material comprises mesonaphthobianthrone.

Optionally in any embodiment the piezochromic material comprises xanthylidene anthrone.

Optionally in any embodiment the piezochromic material comprises dixanthylene.

Optionally in any embodiment the piezochromic material comprises helianthrone.

Optionally in any embodiment the piezochromic material comprises a piezochromic compound having the formula: CuMo₁₋ₓWₓO₄.

Optionally in any embodiment the wall comprises at least one resin selected from a polyester, a polyolefin, and a combination of two or more of these.

Optionally in any embodiment the wall comprises a polyester.

Optionally in any embodiment the wall comprises polyethylene terephthalate.

Optionally in any embodiment the wall comprises polyethylene naphthalate.

Optionally in any embodiment the wall comprises a polyolefin.

Optionally in any embodiment the wall comprises a cyclic olefin copolymer (COC).

Optionally in any embodiment the wall comprises a cyclic olefin polymer (COP).

Optionally in any embodiment the wall comprises a hydrogenated polystyrene.

Optionally in any embodiment the wall comprises a hydrogenated styrene-butadiene copolymer.

Optionally in any embodiment the wall comprises polypropylene.

Optionally in any embodiment at least a portion of the coating or layer has the ratio of elements: SiOₓC_{y} on at least a portion of the interior surface, in which x is from about 0.5 to about 2.9 and y is from about 0.6 to about 3.

Optionally in any embodiment at least a portion of the coating or layer is a gas barrier coating or layer.

Optionally in any embodiment the coating or layer is a coating or layer of SiOₓ, where x is from about 1.5 to about 2.9, alternatively from about 1.5 to about 2.6

Optionally in any embodiment a pharmaceutical preparation, such as an injectable drug, is disposed in the lumen.

Optionally in any embodiment at least a portion of the piezochromic material is at least substantially transparent before at least a portion of the wall is exposed to mechanical stress exceeding the threshold intensity.

Optionally in any embodiment at least a portion of the piezochromic material is at least substantially water white before at least a portion of the wall is exposed to mechanical stress exceeding the threshold intensity.

Optionally in any embodiment at least a portion of the piezochromic material changes its appearance by developing or changing color after at least a portion of the wall is exposed to the mechanical stress exceeding the threshold intensity.

Optionally in any embodiment the color of at least a portion of the piezochromic material is a color other than water white after at least a portion of the wall is exposed to the mechanical stress exceeding the threshold intensity.

Optionally in any embodiment the color of at least a portion of the piezochromic material is blue after at least a portion of the wall is exposed to the mechanical stress exceeding the threshold intensity.

Optionally in any embodiment the color of at least a portion of the piezochromic material is green after at least a portion of the wall is exposed to the mechanical stress exceeding the threshold intensity.

Optionally in any embodiment at least a portion of the wall is water white before at least a portion of the wall is exposed to mechanical stress exceeding a threshold intensity.

Optionally in any embodiment at least a portion of the wall is amber before at least a portion of the wall is exposed to mechanical stress exceeding a threshold intensity.

Optionally in any embodiment at least a portion of the wall is transparent before the wall is exposed to mechanical stress exceeding a threshold intensity.

Optionally in any embodiment at least a portion of the coating or layer on the interior surface is water white before the wall is exposed to mechanical stress exceeding a threshold intensity.

Optionally in any embodiment at least a portion of the coating or layer on the interior surface is transparent before the wall is exposed to mechanical stress exceeding a threshold intensity.

Optionally in any embodiment the change of appearance is detectable using a spectrophotometer.

Optionally in any embodiment the change of appearance is detectable by the eye of a human observer.

Optionally in any embodiment the change of appearance is detectable by the unaided eye of a human observer.

Optionally in any embodiment the threshold intensity is lower than the intensity necessary to damage the coating.

Optionally in any embodiment inspecting the vessel is carried out at last partially by using a spectrophotometer to determine the change in the appearance of at least some of its piezochromic material.

Optionally in any embodiment inspecting the vessel is carried out at least partially using visual inspection to determine the change in the appearance of at least some of its piezochromic material.

Optionally in any embodiment inspecting the vessel is carried out using both visual inspection and a spectrophotometer to determine the change in the appearance of at least some of its piezochromic material.

Optionally in any embodiment the inspecting step is carried out while the vessel is closed.

### BRIEF DESCRIPTION OF THE DRAWINGS

FIG. 1 is a perspective view of a vessel forming one embodiment of the present invention.

FIG. 2 is a similar view of a vessel forming another embodiment of the present invention.

FIG. 3 is a similar view of a vessel forming still another embodiment of the present invention.

FIG. 4 is a similar view of a syringe forming even another embodiment of the present invention.

FIG. 5 is a similar view of a syringe forming yet another embodiment of the present invention.

FIG. 6 is a similar view of a syringe forming another embodiment of the present invention.

### DEFINITION SECTION

In the context of the present specification, the following definitions and abbreviations are used:

The term “at least” in the context of the present invention means “equal or more” than the integer following the term. The word “comprising” does not exclude other elements or steps, and the indefinite article “a” or “an” does not exclude a plurality unless indicated otherwise.

A “vessel” in the context of the present invention can be any type of vessel with at least one opening and a wall defining an interior surface. The term “at least” in the context of the present invention means equal to or more than the number following the term. Thus, a vessel in the context of the present invention has one or more openings. One or two openings, like the openings of a sample tube (one opening) or a syringe barrel (two openings) are preferred. If the vessel has two openings, they can be of same or different size. If there is more than one opening, one opening can be used for the gas inlet for a PECVD coating method, while the other openings are either capped or open. A vessel according to the present invention can be a sample tube, e.g. for collecting or storing biological fluids like blood or urine, a syringe (or a part thereof, for example a syringe barrel or cartridge) for storing or delivering a biologically active compound or composition, e.g. a medicament or pharmaceutical composition, a vial for storing biological materials or biologically active compounds or compositions, a pipe, e.g. a catheter for transporting biological materials or biologically active compounds or compositions, or a cuvette for holding fluids, e.g. for holding biological materials or biologically active compounds or compositions.

A vessel can be of any shape, a vessel having a generally cylindrical wall being preferred. Generally, the interior wall of the vessel is cylindrically shaped, as in a sample tube or a syringe barrel. Sample tubes and syringes or their parts (for example syringe barrels or cartridges) are contemplated.

A barrier coating or layer is a coating or layer on a substrate that provides a positive barrier improvement factor (BIF) greater than one, compared to the same substrate but without the barrier coating or layer. A BIF can be determined, for example, by providing two groups of identical substrates, adding a barrier layer to one group of substrates, testing a barrier property (such as the rate of outgassing or leaching of contents of the vessel or the rate of ingress of some material, for example, air, oxygen, moisture, or other external constituents, all broadly referred to as transfer rates) on the substrates having a barrier, doing the same test on substrates lacking a barrier, and taking a ratio of the transfer rate of the material with versus without a barrier. For example, if the rate of outgassing of material through the barrier is one-third the rate of outgassing of the same material without a barrier, the barrier has a BIF of 3. A barrier coating or layer can be independently applied or formed by modification of a preexisting layer.

SiOx refers to a material necessarily containing silicon (Si) and oxygen (O) in the atomic ratio expressed by the defined value(s) of x, optionally further containing any additional elements. The value of x can be integral or non-integral and SiOₓ does not need to be a stoichiometric compound, a complete compound, or a single compound.

SiOxCy refers to a material necessarily containing Si, O, and carbon (C) in the atomic ratio expressed by the defined value(s) of x and y, optionally further containing any additional elements. The values of x and y can be integral or non-integral and SiOxCy does not need to be a stoichiometric compound, a complete compound, or a single compound.

Chemical vapor deposition (CVD) is a process in which one or more precursors is supplied as a gas to the vicinity of a surface. A gas phase reaction occurs near or on the surface, changing the composition of at least one precursor and depositing the changed composition as a layer on the surface. CVD typically is used to deposit a very thin layer less than one micron (10⁻⁶ meters) thick.

Plasma enhanced chemical vapor deposition (PECVD) is chemical vapor deposition in which a plasma is also formed at the site of reaction by suitable apparatus, typically a radio frequency or microwave energy applicator. PECVD apparatus and processes are described in U.S. Pat. No. 7,985,188, for example.

The thickness of a PECVD layer is the thickness as measured by transmission electron microscopy (TEM), for example as described in U.S. Pat. No. 7,985,188.

A mechanical stress of “threshold intensity” is defined as the maximum stress or force that can be applied under test or operational conditions, to an article having piezochromic material, that does not cause the piezochromic material to change its appearance. The article having piezochromic material usefully is designed to have a threshold intensity that is no greater than the minimum intensity that would cause the article to be rejected as defective or possibly defective due to excessive experienced stress (desirably allowing a sufficient safety factor). Then, if an article so designed is interrogated and exhibits a change of appearance, this change of appearance indicates that it has experienced a stress exceeding its threshold intensity. The article can be rejected based on detection of the change of appearance.

An article “having the property of changing its appearance” is defined as an article that will change its appearance if it experiences the stated stress exceeding its threshold intensity, whether or not the stress has actually been applied at the time in question. “Change in appearance” is flexibly defined, and includes, for example, the intensity of color, hue of color, a change in the absorbance, transmission, radiation, or reflection of a selected wavelength of energy, or a difference in the degree of transparency, whether detectable by a machine or the aided or unaided human eye. “Color” is also broadly defined to include black, white, and shades of gray; the usual primary and mixed colors of pigments or radiation; and frequencies or mixtures of frequencies of radiation either visible or non-visible to the human eye, thus including visible light, infrared light, ultraviolet light, and other electromagnetic energy. A “change of color” includes both a change from one color to another and a change from colorless to colored or vice versa.

“Transparent” is defined as a material or article transmitting a detectable image, whether or not the true color of the image is modified. For example, an amber colored vial through which the contents can be viewed, but appear to be amber when so viewed, is transparent as defined here.

“Water white” means transparent and colorless. It is not an absolute term, as few if any articles are absolutely transparent or colorless. It is a term used in the pharmaceutical industry, for example, to indicate a container that appears colorless and transparent.

### DETAILED DESCRIPTION OF THE PREFERRED EMBODIMENTS

The present invention will now be described more fully hereinafter with reference to the accompanying drawings, in which one or more embodiments of the invention are shown. This invention may, however, be embodied in many different forms and should not be construed as limited to the embodiments set forth herein. Rather, these embodiments are examples of the invention, which has the full scope indicated by the language of the claims. Like numbers refer to like elements throughout.

One aspect of the present technology is a way to assess the strain imparted on a nano-thin plasma barrier coated plastic vessel, for example a medical device or vessel, by associating the vessel with a piezochromic indicator. Using this technology, the strain history of the article can be determined at some point after the strain has occurred. This technology finds particular utility when the strain imparted on the article is not detectable by looking for changes in the article itself.

A mechanical strain on the article can result in either a reversible unchanged dimensional article or irreversible dimensionally changed article. The most important aspect is the former, when a strain is imparted but not manifested by a dimensional change in the article itself. Additional stress on the already-strained article can lead to permanent (irreversible) article deformation, including catastrophic failure (fracture), yielding or drawing. In other words, after an initial strain the article may be damaged, but not to the point that the damage can be detected, at least readily detected, by examining the article itself. The latter situation, where the article is visibly damaged, is less of an issue if the damage can be readily detected with simple visual or metrology methods.

It is important to detect the initial strain when very little value has been added to the article. For example when a pharmaceutical vial has been damaged but the damage is not yet evident, it is important to detect the initial damage and remove the damaged vial from further processing, particularly before it is filled with an expensive pharmaceutical preparation.

It is also useful to detect any strains at the end of a manufacturing operation, as when the filled pharmaceutical packages are to be shipped from the filling plant, so the articles can be audited for any processing damage at the end of the process. If the articles pass inspection at this stage, and subsequently are found to be defective, evidence has been created that the damage happened after the end-of-manufacturing inspection.

Additionally, it is useful to detect any historical strains in manufactured articles as received, so the receiving party can ascertain whether it has received damaged goods.

The piezochromic indicator can be adapted so that when the article is strained but then returns to its original dimensions, the piezochromic indicator associated with the vessel is irreversibly changed or at least changed in a way that is detectable for a period of time after it occurs, optionally for at least some period after the vessel returns to its original dimensions.

The change in question can be, for example, a change in radiation absorption (e.g. colorometric in visible range) when a mechanical stress is imparted on the article.

Certain dyes within polymers are known to respond to specific stimuli and indicate exposure to stimuli by a change or shift in the frequencies of light which they absorb. The stimuli include temperature, radiation, chemicals (e.g. H₂O, CO₂, NO₂, ethylene, and SO₂), and strain. In one manifestation, irreversible piezochromic (alternatively tribochromic) dyes can be incorporated with the plastic article, and a (color) shift in light absorption (frequency) can indicate both the location and extent of any significant strain imparted on the plastic part.

The wavelengths of light desirably absorbed are from about 10 nm to about 1 mm which include ultraviolet, visible and infrared. More desirably, one or more frequencies of absorbed light, which shift on exposure to the stimuli, are in the visible light region which is from about 0.4 microns to about 0.7 microns. Exposure of the dye to its specific stimuli causes a change in the dye which causes a change in the amount of one or more frequencies of light which the dye absorbs. These shifts are usually characterized by a spectrometer which measures the amount of absorbed or reflected light from a material at numerous different frequencies. Many of these dyes after exposure to their specific stimuli undergo a large enough shift in one or more frequencies of visible light absorbed by the dye that the exposure to the stimuli can be detected by a person as a change in the perceived color of the dye. Through prior calibration of dye color shift to plasma coating or layer failure on the vial, an inline light frequency sensor could rapidly detect color shifts and permit removal of strained plastic articles.

The contemplated piezochromic dyes include, but are not limited to, those defined in U.S. Pat. No. 5,501,945. Several suitable examples follow.

a. Triaryl imidazole dimers of Bis-2,4,5-triaryl imidazoles having one or more substituents groups selected from aryl groups such as phenyl, p-tolyl, p-chlorophenyl, p-anisyl. Preferred are: 2,2′,4,4′5,5′-hexaphenyl bisimidazole; 2,2′,4,4′5,5′-hexa-p-tolyl bisimidazole; 2,2′,4,4′5,5′-hexa-p-chlorophenyl bisimidazole; 2,2′-di-p-chlorophenyl-4,4′,5,5′ tetraphenyl bisimidazole; 2,2′-di-p-Anisyl-4,4′,5,5′-tetraphenyl bisimidazole; and 2,2′-di-p-tolyl-4,4′,5′-tetraphenyl bisimidazole.

b. Bis-tetraaryl pyrrole. Preferred is: Bis-tetra phenyl pyrrole.

c. Bianthrones: Δ10,10′-bianthrone, Preferred is 2,4,2′,4′-tetramethyl bianthrone.

d. Xanthylidene anthrone.

e. Dixanthylene.

f. Helianthrone.

g. Mesonaphthobianthrone.

Additionally, certain inorganic materials may also function as piezochromic materials, as described in Characterization of the Piezochromic Behavior of Some Members of the CuMo₁₋ₓWₓO₄ Series, Inorg. Chem., 2008, 47 (7), pp 2404-2410. The piezochromic materials of the just-cited article are incorporated here by reference.

Any polymer suitable for making an article to be treated with piezochromic material can be used. For example, polymer types contemplated for use in the present technology include at least one resin selected from polyesters, polyolefins, modified polystyrenes, polystyrene-polybutadiene copolymers, and a combination of two or more of these. The polyesters contemplated for the present use include polyethylene terephthalate or polyethylene naphthalate. The polyolefins contemplated for the present use include cyclic olefin polymer, cyclic olefin copolymer, or polypropylene.

One particular type of polymer contemplated for use in the present technology is cyclic olefin polymer (COP). COP can be manufactured using a catalytic ring opening metathesis polymerization (ROMP) process involving (co)polymerization of one or more norbornene monomers followed by catalytic hydrogenation to a saturated bicyclic backbone structure. Some examples of typical commercial COP resins useful for medical device manufacture are Zeonex 690r, Zeonex 790r, Zeonor 1020r, Zeonor 1060r, Zeonor 1420, and Zeonor 1600, and Crystal Zenith, produced by Zeon Chemicals, L.P.

Another type of polymer contemplated for use in the present technology is modified polystyrene. One example of a modified polystyrene is hydrogenated polystyrene (alternatively poly(cyclohexylethylene) (PCHE). PCHE is manufactured by catalytic heterogeneous hydrogenation of polystyrene. Using narrow molecular weight polystyrene derived from anionic polymerization of styrene, PCHE polymers with glass transition temperatures (T_{g}) as high as 148° C. can be realized. The Dow Chemical Company has produced this material.

More examples of suitable modified polystyrenes are hydrogenated styrene-butadiene copolymer (SBC) and hydrogenated styrene-butadiene-styrene triblock copolymer. These copolymers are manufactured by anionic polymerization of butadiene and styrene monomers, followed by butadiene double bond hydrogenation. The reaction conditions are such that the styrene ring remains unsaturated. Typical commercial SBCs useful for medical device manufacture are K-Resin SBC BK10, K-Resin SBC KR01, K-Resin SBC KR03, K-Resin SBC KR03NR, and K-Resin SBC XK44, produced by Chevron Phiiips Chemical Company, LLC.

Plastic vials, syringe barrels, sample collection tubes, other types of medical vessels and devices can be manufactured by injection molding, blow molding, or otherwise forming articles from molding compositions containing these resins.

One method of forming medical vessels or other articles modified with piezochromic material is incorporation of a piezochromic indicator (additive/coating) in the resin composition used to make the article, then molding or otherwise forming the plastic/SiOx molded laminate article from the modified resin.

Alternatively, already-formed articles can be treated following molding to provide the photochromic material. Not to be limiting, incorporation of the piezochromic dyes could be in one or more modes:
coated over outside of plastic vial
solvent-absorbed into exterior of plastic vial
coated over inside of plastic vial before plasma coating
solvent-absorbed into interior of plastic vial before plasma coating.

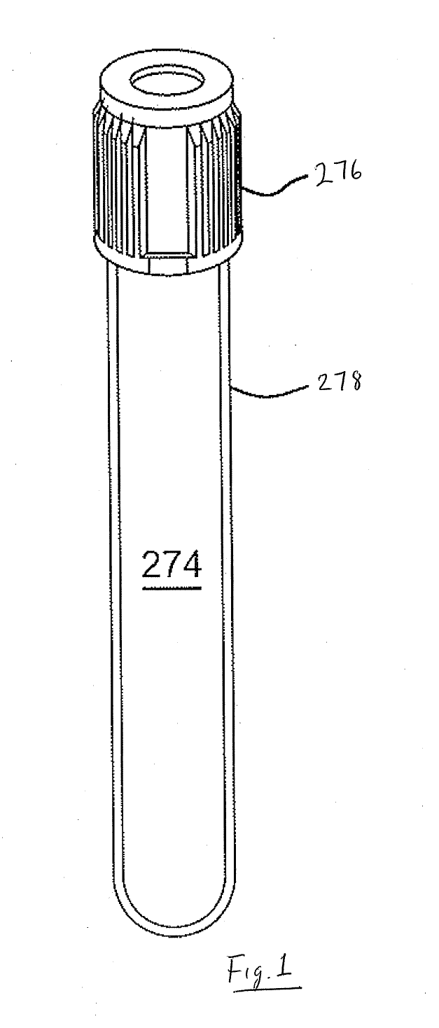

Referring to the drawing figures, FIG. 1 shows a vessel 274 having a cap 276 and a wall 278. The wall 278 incorporates as a homogeneous part of the resin composition a piezochromic material, which can be any one or more of such materials described in this disclosure. The piezochromic material is selected and used in such a way, as by using a suitable proportion, to change in appearance when subjected to a stress exceeding a threshold. For example, one useful threshold is the amount of piezochromic material necessary to change appearance when the vessel is bent sufficiently to crack the vessel itself.

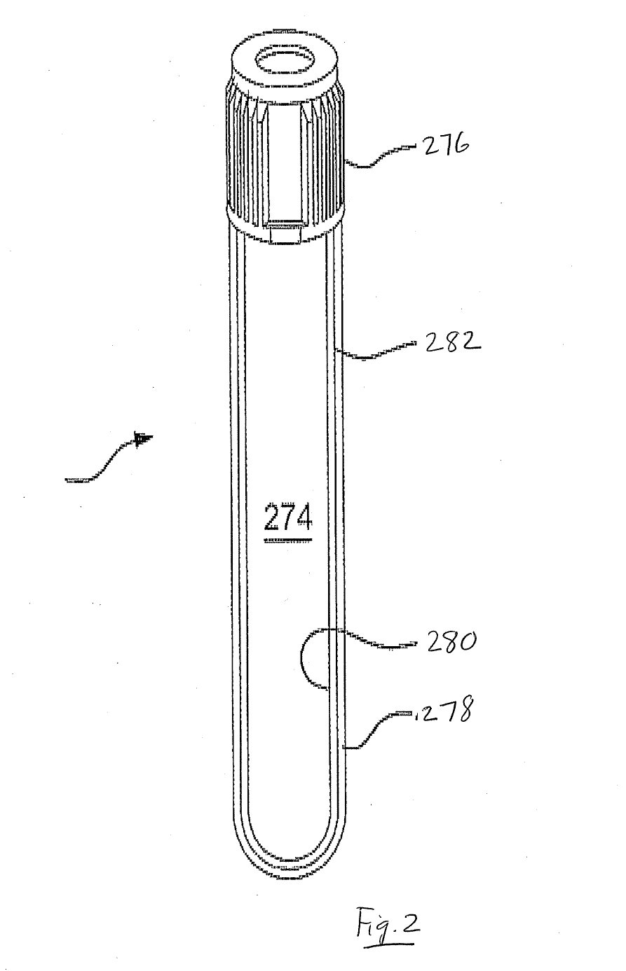

FIG. 2 shows a similar vessel further including a coating or layer 280 on the interior surface 282 having the ratio of elements SiOₓ, in which x in this formula is from about 1.5 to about 2.9, the coating or layer having a thickness of less than 200 nm. The coating or layer thickness is not critical, although individual SiOₓ barrier layers applied by plasma-enhance chemical vapor deposition or other techniques are usually this thin or thinner. Alternatively, at least a portion of the coating or layer can have the ratio of elements: SiOₓC_{y} on at least a portion of the interior surface, in which x is from about 0.5 to about 2.9 and y is from about 0.6 to about 3. The application of such coating or layers is described, for example, in U.S. Published Patent Application 2010/0298738, which is hereby incorporated by reference in its entirety. The piezochromic material is selected and used in the vessel wall 278 in such a way, as by using a suitable proportion, to change in appearance when at least a portion of the coating or layer 280 is subjected to a stress exceeding a threshold. For example, one useful threshold is the amount of piezochromic material necessary to change appearance when the vessel is bent sufficiently to crack or reduce the barrier efficacy of at least a portion of the coating or layer 280.

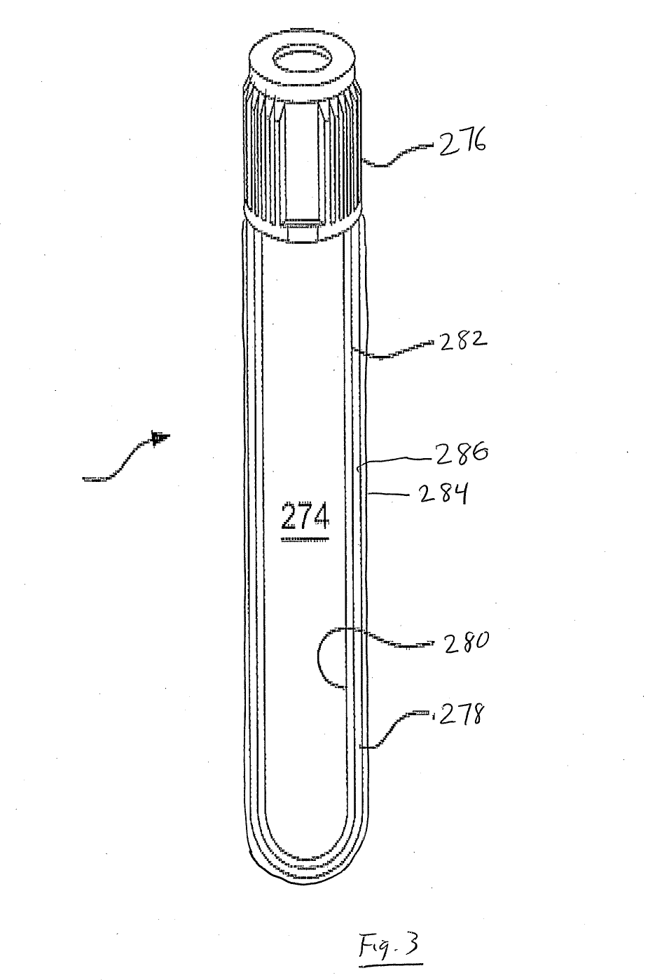

FIG. 3 shows a similar vessel 274, except further including a coating or layer 284 of piezochromic material, and either including or free of piezochromic material in the wall 278. The piezochromic material is selected and used in at least a portion of the coating or layer 284 in such a way, as by using a suitable proportion, to change in appearance when at least a portion of the coating or layer 280 is subjected to a stress exceeding a threshold.

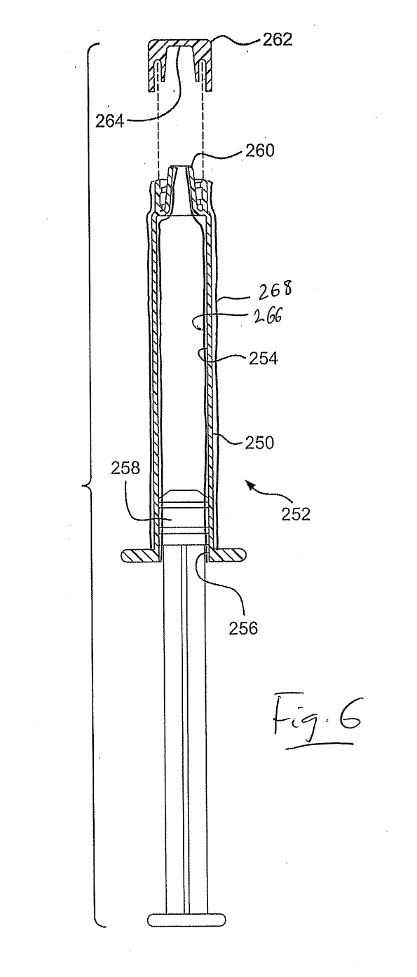

In an alternative embodiment similar to FIG. 3, the piezochromic layer 284 can be provided on the inside of the vessel wall 278, for example between the vessel wall 278 and the barrier coating or layer 280. This construction has the advantage of protecting the piezochromic layer from scratches and other minor insults that might be sufficient to trigger a appearance change but insufficient to damage the interior barrier coating or layer 280 or vessel wall 278. Sandwiching the piezochromic layer between the barrier coating or layer 280 and vessel wall 278 protects the piezochromic layer from oxygen and other environmental agents in the atmosphere. The barrier coating or layer 280 also protects the piezochromic layer from the contents of the vessel and vice versa. The same modification is contemplated for the embodiment of FIG. 6 as discussed below.

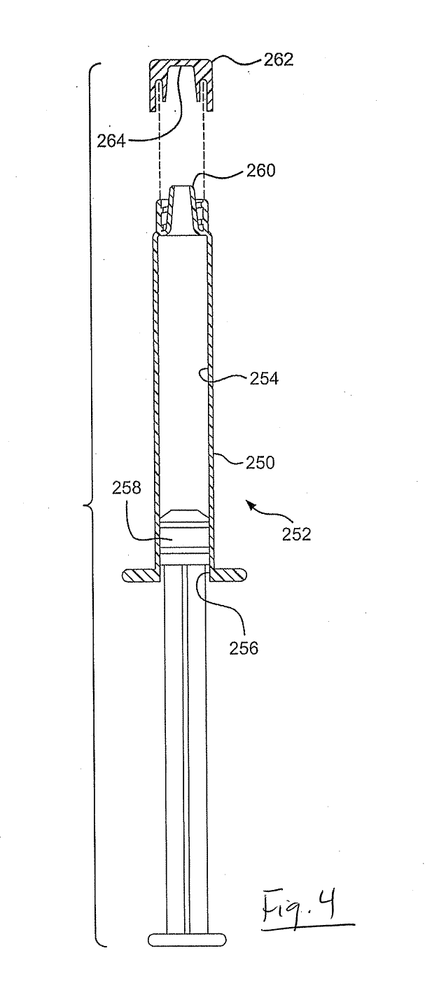

FIGS. 4-6 are analogous to FIGS. 1-3, but show as a more specific embodiment a syringe 252 having a syringe barrel 250 defining a vessel wall, an inner surface 254 of the barrel 252, an opening 256 closed by a plunger 258, and a Luer fitting 260 defining an opening that can be closed by a web 264 of the cap 262. The cap 262 can be removed and replaced by a hypodermic needle to inject the contents of the lumen defined within the surface 254, which can be a pharmaceutical preparation, into a subject or medical apparatus. The illustrated embodiment, supplied with a single dose of a drug, is commonly referred to as a prefilled syringe.

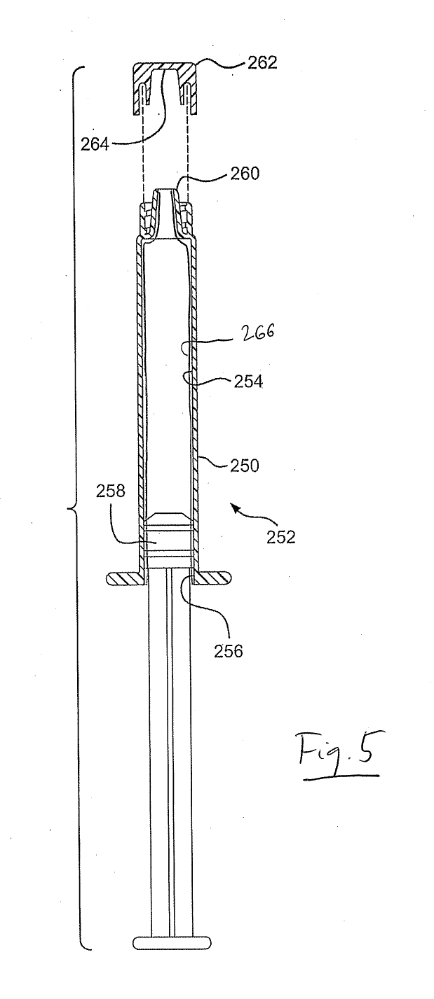

The vessel of FIG. 4 has an uncoated barrel 254 containing a homogeneously dispersed piezochromic material in its material. FIG. 5 differs in from FIG. 4 in that FIG. 5 has an inner coating or layer 266 of SiOx. FIG. 6 differs in from FIG. 5 in that FIG. 6 has an outer coating or layer 268 of piezochromic material. In FIG. 6, the barrel 250 can either contain or not contain a dispersed piezochromic material in its material.

Various formulations and methods of piezochromic dyes can be incorporated with the plastic articles, including plastic dispersions including master batches, latex, paints, or inks which can be coated or absorbed (melt, spray, dip).

The amount of the above described dyes to be used in polymeric compositions is desirably from 0.001 to 5 weight percent based on the portion of the polymeric composition containing the dye. More desirably the amount is from 0.01 to 5 weight percent and preferably from 0.1 to 1 weight percent. If the polymeric composition includes a non-reactive diluent or solvent that will be removed, the weight percent dye is to be calculated based on the composition less the diluent or solvent.

### Prophetic Example

The exterior of a molded TOPAS 6013 resin five milliliter vial, with an internally coated SiOₓ plasma barrier coating or layer on a round cylindrical wall, is immersed into a one percent solution of 2,2′,4,4′,5,5′-hexa-p-tolyl bisimidazole warmed (60° C.) toluene solution for one hour, then removed and placed in a drying oven for one hour.

The vial is then placed in a UV-Visible spectrophotometer and the spectrum recorded. The unstressed spectrum indicates a pale-green color.

The vial is then placed on its side in a mechanical vise and the vise is compressed until a radial deformation of 10 percent is realized. This deformation is defined as the increase in radius at the point of deformation. The vial is then removed from the vise and the UV-Visible spectrum is re-measured in the UV-Visible spectrophotometer. There has been a shift of the spectrum indicating a blue color. The vial thus bears a detectable indication that it has been deformed to a degree sufficient to trigger the piezochromic layer.

1. A medical device comprising:
a wall having an interior surface defining a lumen and an exterior surface;
a coating or layer on the interior surface; and
a piezochromic material associated with the wall, the piezochromic material having the property of changing its appearance when the wall is exposed to mechanical stress exceeding a threshold intensity.

2. The medical device of claim 1, in which at least a portion of the coating or layer is a barrier coating or layer.

3. The medical device of claim 1, in which at least a portion of the coating or layer has the ratio of elements SiOx, in which x in this formula is from about 1.5 to about 2.9.

4. The medical device of claim 1, in which at least a portion of the coating or layer is applied using chemical vapor deposition.

5. The medical device of claim 1, in which at least a portion of the coating or layer is applied using plasma enhanced chemical vapor deposition

6. (canceled)

7. The medical device of claim 1, in which at least a portion of the wall is comprised of thermoplastic material.

8. The medical device of claim 1, in which the piezochromic material is coated on at least a portion of the exterior surface.

9. (canceled)

10. The medical device of claim 1, in which the piezochromic material is a layer between the interior surface of the wall and at least a portion of the coating or layer.

11. The medical device of claim 1, in which the piezochromic material is incorporated in the wall.

12. The medical device of claim 1, in which the piezochromic material is homogeneously incorporated in the wall.

13. The medical device of claim 1, in which the piezochromic material comprises:
a triaryl imidazole dimer of bis-2,4,5-triaryl imidazole;
bis-tetraaryl pyrrole;
a bianthrone;
xanthylidene anthrone;
dixanthylene;
helianthrone;
a piezochromic compound having the formula: CuMo1-xWxO4; or
a combination of two or more of these.

14-15. (canceled)

16. The medical device of claim 1, in which the piezochromic material comprises:
2,2′,4,4′5,5′-hexaphenyl bisimidazole;
2,2′,4,4′,5,5′-hexa-p-tolyl bisimidazole;
2,2′,4,4′,5,5′-hexa-p-chlorophenyl bisimidazole;
2,2′-di-p-chlorophenyl-4,4′,5,5′tetraphenyl bisimidazole;
2,2′-di-p-anisyl-4,4′,5,5′-tetraphenyl bisimidazole;
2,2′-di-p-tolyl-4,4′,5,5′-tetraphenyl bisimidazole or
a combination of two or more of these.

17-26. (canceled)

27. The medical device of claim 1, in which the wall comprises at least one resin selected from a polyester, a polyolefin, and a combination of two or more of these.

28-38. (canceled)

37. The medical device of claim 1, in which at least a portion of the coating or layer has the ratio of elements: SiOxCy on at least a portion of the interior surface, in which x is from about 0.5 to about 2.9 and y is from about 0.6 to about 3.

38. (canceled)

39. The medical device of claim 1, in which at least a portion of the coating or layer has the ratio of elements: SiOx, in which x is from about 1.5 to about 2.9.

40. (canceled)

41. The medical device of claim 1, further comprising a pharmaceutical preparation disposed in the lumen.

42. (canceled)

43. The medical device of claim 1, in which at least a portion of the piezochromic material is at least substantially water white before at least a portion of the wall is exposed to mechanical stress exceeding the threshold intensity.

44-57. (canceled)

58. The medical device of claim 1, in which the medical device is a vial, syringe barrel, auto-injector cartridge, sample collection tube, or micro-titer plate.

59. A method of interrogating a medical device for processing damage, comprising:
providing a medical device of claim 1;
inspecting the medical device from the exterior for a change in the appearance of at least some of its piezochromic material that is characteristic of exposure of the wall to mechanical stress exceeding the threshold intensity greater than zero.

60-63. (canceled)

64. The medical device of claim 1, in which the piezochromic material comprises: 2,2′,4,4,′5,5′-hexa-p-tolyl bisimidazole.
